(19) United States
(12) Patent Application Publication
(10) Pub. No.: US 2021/0053954 A1
(43) Pub. Date: Feb. 25, 2021

(54) AROMATIC AMINE COMPOUND, COVERING LAYER MATERIAL, AND LIGHT-EMITTING ELEMENT

(71) Applicant: Toray Industries, Inc., Tokyo (JP)

(72) Inventors: Fangzhu Sun, Minhang District Shanghai (CN); Peng Wang, Minhang District Shanghai (CN); Guangnan Jin, Minhang District Shanghai (CN); Jincai Li, Minhang District Shanghai (CN)

(73) Assignee: Toray Industries, Inc., Tokyo (JP)

(21) Appl. No.: 16/961,513

(22) PCT Filed: Jan. 15, 2019
(86) PCT No.: PCT/CN2019/071782

§ 371 Date: Jul. 10, 2020

(30) Foreign Application Priority Data
Jan. 31, 2018 (CN) 201810094263.1

(51) Int. Cl. C07D 409/02; C09K 11/06; H01L 51/52; H01L 51/50

(52) U.S. Cl. CPC C07D 409/02; C09K 11/06; C09K 2211/1029; H01L 51/5012; H01L 51/5237

(57) ABSTRACT

The present invention provides an aromatic amine compound as represented by formula (1) for improving the light extraction efficiency and color purity of an organic light-emitting element, an organic light-emitting element material containing the aromatic amine compound, a covering layer material of organic light-emitting element, and an organic light-emitting element.

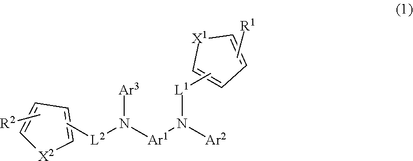

The organic light-emitting element provided by the present invention can achieve high luminous efficiency and color reproducibility. The organic light-emitting element of the present invention can be used for an organic EL display, a backlight source of a liquid crystal display, illumination, light sources for gauges, a sign board, a marker light, etc. The present invention provides an organic light-emitting element having greatly improved light extraction efficiency and excellent color purity.

### CROSS REFERENCE TO RELATED APPLICATIONS

This is the U.S. National Phase application of PCT/CN2019/071782, filed Jan. 15, 2019, which claims priority to Chinese Patent Application No. 201810094263.1, filed Jan. 31, 2018, the disclosures of each of these applications being incorporated herein by reference in their entireties for all purposes.

### FIELD OF THE INVENTION

The present invention relates to a novel aromatic amine compound for an organic light-emitting element, a covering layer material containing the aromatic amine compound, and a light-emitting element, and more particularly relates to an aromatic amine compound for organic light-emitting element with greatly improving the light extraction efficiency, a covering layer material and a light-emitting element.

### BACKGROUND OF THE INVENTION

An organic light-emitting element is a self-luminous display device, which has the characteristics of lightweight, small thickness, wide viewing angle, low power consumption, high contrast, and the like.

The principle of light emission of the organic light-emitting element lies in that light is generated when holes and electrons injected from an electrode return to a ground state from an excited state by recombination in a light-emitting layer. This light-emitting element has the characteristic of small thickness and capable of emitting light at a high brightness under a low driving voltage and capable of emitting light with a plurality of colors by selecting light-emitting materials, and thus it has attracted much attention.

Since C. W. Tang et al. of Kodak Co., Ltd. has revealed that organic thin film elements can emit light at high brightness, many studies have been conducted on their applications. The organic thin film light-emitting elements are used in main display screens of mobile phones, and achieve realistic progress in terms of practicality. However, there are still many technical topics, among which high efficiency and low power consumption of elements are major topics.

The organic light-emitting elements may be classified into bottom-emission organic light-emitting elements and top-emission organic light-emitting elements according to a direction in which light generated by an organic light-emitting layer is emitted. In a bottom-emission organic light-emitting element, light is emitted towards the substrate side, a reflective electrode is formed on the upper part of the organic light-emitting layer, and a transparent electrode is formed on the lower part of the organic light-emitting layer. In this case, when the organic light-emitting element is an active matrix element, the light-emitting area is reduced because a portion in which a thin film transistor is formed is opaque. On the other hand, in a top-emission organic element, a transparent electrode is formed on the upper part of an organic light-emitting layer, and a reflective electrode is formed on the lower part of the organic light-emitting layer, so that the light is emitted in a direction opposite to the substrate side. Therefore, an area through which light passes is increased and the brightness rises.

In the prior art, in order to improve the luminous efficiency of the top-emission organic light-emitting element, a method is adopted to form an organic covering layer on an upper translucent metal electrode through which the light of the light-emitting layer passes, so as to adjust an optical interference distance, and suppress external light reflection and extinction caused by surface plasma energy movement, and the like (see Patent Documents 1 to 6).

For example, as described in Patent Document 2, an organic covering layer having a refractive index of 1.7 or more and a film thickness of 600 Å is formed on an upper translucent metal electrode of a top-emission organic light-emitting element, such that the luminous efficiency of organic light-emitting elements that emit red light and green light is improved by about 1.5 times. The adopted material of the organic covering layer is an amine derivative, a quinolinol complex or the like.

As described in Patent Document 4, a material with an energy gap of less than 3.2 eV will affect the blue wavelength and is not suitable for the use in an organic covering layer, and the adopted organic covering layer material is an amine derivative having a specific chemical structure, or the like.

As described in Patent Document 5, in order to realize a blue light-emitting element having a low CIEy value, a refractive index variation of an organic covering layer material at a wavelength of 430 nm to 460 nm is Δn>0.08, and the adopted organic covering layer material is an anthracene derivative having a specific chemical structure, or the like.

As described in Patent Document 6, it is possible to obtain an organic light-emitting element having greatly improved light extraction efficiency and excellent color purity after organic coating materials, such as thiophene and pyrrole, are used.

### PATENT DOCUMENTS

Patent Document 1: WO2001/039554

Patent Document 2: JP Laid-open 2006-156390

Patent Document 3: JP Laid-open 2007-103303

Patent Document 4: JP Laid-open 2006-302878

Patent Document 5: WO2011/043083

Patent Document 6: CN104744450A

### SUMMARY OF THE INVENTION

As described above, in the prior art, an amine derivative having a specific structure and a high refractive index or a material satisfying specific parameters is used as the organic coating layer material to improve the light extraction efficiency and color purity, but the problem of achieving both the high luminous efficiency and the high color purity has not been solved yet, especially in the case of preparing a blue light-emitting element.

The present invention provides an aromatic amine compound for improving the light extraction efficiency and color purity of an organic light-emitting element, an organic light-emitting element material containing the aromatic amine compound, a covering layer material of the organic light-emitting element, and an organic light-emitting element.

The aromatic amine compound provided by the present invention has excellent thin-film stability and high refractive index, since it has a thiophene structure, a furan structure or a pyrrole structure, and can solve the problems of improving the extraction efficiency and color purity simultaneously.

In exemplary embodiments of the present invention, the structure of the aromatic amine compound is specifically represented by the following formula (1).

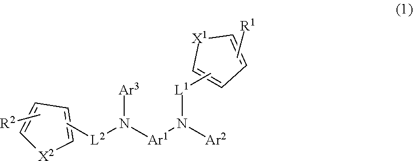

wherein, X¹ and X² are selected from sulfur atoms, oxygen atoms or N—R, wherein R is independently selected from one or more of the group consisting of hydrogen, deuterium, optionally substituted alkyl group, optionally substituted cycloalkyl group, optionally substituted heterocyclic group, optionally substituted alkenyl group, optionally substituted cycloalkenyl group, optionally substituted alkynyl group, optionally substituted alkoxyl group, optionally substituted alkyl sulphanyl group, optionally substituted aryl ether group, optionally substituted aryl thioether group, optionally substituted aryl group, optionally substituted heteroaryl group, optionally substituted carbonyl group, optionally substituted carboxyl group, optionally substituted oxycarbonyl group, optionally substituted carbamoyl group, optionally substituted alkylamino group, or optionally substituted silanyl group;

L¹ and L² may be identical or different, and are independently selected from one of arylene group, heteroarylene group or direct bonding;

Ar¹ is selected from arylene group;

Ar² and Ar³ may be identical or different heteroaryl groups;

wherein, R¹ and R² may be identical or different, and are independently selected from one or more of the group consisting of hydrogen, deuterium, halogen, optionally substituted alkyl group, optionally substituted cycloalkyl group, optionally substituted heterocyclic group, optionally substituted alkenyl group, optionally substituted cycloalkenyl group, optionally substituted alkynyl group, optionally substituted alkoxyl, optionally substituted alkyl sulphanyl group, optionally substituted aryl ether group, optionally substituted aryl thioether group, optionally substituted aryl group, optionally substituted heteroaryl group, optionally substituted cyano group, optionally substituted carbonyl group, optionally substituted carboxyl group, optionally substituted oxycarbonyl group, optionally substituted carbamoyl group, optionally substituted alkylamino group, or optionally substituted silanyl group; or may also be bonded with adjacent substituents to form a ring.

Based on the thermal stability of the compound and the effect on the light extraction efficiency, in the formula (1), it is preferable that R¹ and R² are one or more of optionally substituted aryl group or heteroaryl group.

Based on the introduction of heteroatoms, the refractive index of the compound may be increased. Preferably, the X¹ and X² are selected from sulfur atoms, L¹ and L² are selected from arylene group, and R¹ and R² are aryl group.

Based on the synthesability of the material, in the formula (1), preferably, alkyl group is C1-C20 alkyl group; cycloalkyl group is C3-C20 cycloalkyl group; heterocyclic group is C2-C20 heterocyclic group; alkenyl group is C2-C20 alkenyl group; cycloalkenyl group is C3-C20 cycloalkenyl group; alkynyl group is C2-C20 alkynyl group; alkoxyl group is C1-C20 alkoxyl group; alkyl sulphanyl group is C1-C20 alkyl sulphanyl group; aryl ether group is C6-C40 aryl ether group; the aryl thioether group is C6-C60 aryl thioether group; aryl group is C6-C60 aryl group; and heteroaryl group is C4-C60 aromatic heteroaryl group.

Based on a reduction in the crystallinity of the material, it is preferable that the Ar¹ is non-condensed-ring arylene group.

Based on an improvement in the optical performance of the material, it is preferable that the Ar² and Ar³ are heteroaryl group directly connected to nitrogen. That is, there is no other non-heteroaryl group between the nitrogen atom and the heteroaryl group. These non-heteroaryl groups include, but are not limited to, arylene group.

The present invention in exemplary embodiments further provides an organic light-emitting element material, wherein the material contains the aromatic amine compound. An organic light-emitting element according to embodiments of the present invention comprises: a substrate, a first electrode, a light-emitting layer containing more than one organic layer film, a second electrode, and a covering layer, wherein the organic light-emitting element contains the organic light-emitting element material.

The present invention in exemplary embodiments further provides a covering layer material of organic light-emitting element, wherein the material contains the above aromatic amine compound.

At last, the present invention further provides an organic light-emitting element, comprising: a substrate, a first electrode, no less than one organic layer film including a light-emitting layer, a second electrode element, and also a covering layer, wherein the covering layer contains the covering layer material of the organic light-emitting element.

It can be believed that the mechanism of the present invention is as follows (but the present invention is not bound for any purpose): the aromatic amine compound provided by embodiments of the present invention has excellent thin-film stability and high refractive index, as it has a thiophene structure, a furan structure or a pyrrole structure, and can solve the problems of improving the light extraction efficiency and color purity simultaneously. The compound represented by the formula (1) is used in the covering layer material and has a thiophene structure, a furan structure or a pyrrole structure, so the covering layer material has a high glass transition temperature and a steric hindrance effect, thereby achieving excellent thin-film stability. In addition, the thiophene structure, the furan structure or the pyrrole structure can improve the light absorption coefficient of the compound and obtain a higher attenuation coefficient, and the higher the light absorption coefficient and attenuation coefficient (k) is, the higher the refractive index is. Therefore, a thin film in an ultraviolet and visible range can obtain a higher refractive index. Moreover, heteroaryl has the property of increasing polarizability and can further increase the refractive index.

Therefore, the aromatic amine compound having a high refractive index is used in the covering layer material, thereby obtaining an organic light-emitting element that greatly improves the light extraction efficiency and has excellent color purity.

The alky group is preferably C1-C20 alkyl group, and further preferably one or more of saturated aliphatic hydrocarbon groups, such as methyl group, ethyl group, n-propyl group, isopropyl group, n-butyl group, sec-butyl group, or tert-butyl group. The alkyl group may or may not have a substituent.

The cycloalkyl group is preferably C3-C20 cycloalkyl group, and further preferably one or more of saturated alicyclic hydrocarbon groups, such as cyclopropyl group, cyclohexyl group, norbornyl group, or adamantyl group. The cycloalkyl group may or may not have a substituent.

The heterocyclic group is preferably C2-C20 heterocyclic group, and further preferably one or more of aliphatic rings having atoms other than carbon in a ring, such as a pyran ring, a piperidine ring, or a cyclic amide. The heterocyclic group may or may not have a substituent.

The alkenyl group is preferably C2-C20 alkenyl group, and further preferably one or more of unsaturated aliphatic hydrocarbon groups containing a double bond, such as vinyl group, allyl group, or butadienyl group. The alkenyl group may or may not have a substituent.

The cycloalkenyl group is preferably C3-C20 cycloalkenyl group, and further preferably one or more of unsaturated alicyclic hydrocarbon groups containing a double bond, such as cyclopentenyl group, cyclopentadienyl group, or cyclohexenyl group. The cycloalkenyl group may or may not have a substituent.

The alkynyl group is preferably C2-C20 alkynyl group, and further preferably an unsaturated aliphatic hydrocarbon group containing a triple bond, such as ethynyl group. The alkynyl group may or may not have a substituent.

The alkoxyl group is preferably C1-C20 alkoxyl group, and further preferably one or more of functional groups of aliphatic hydrocarbon groups bonded by an ether bond, such as methoxyl group, ethoxyl group or propoxyl group. The aliphatic hydrocarbon group may or may not have a substituent.

The alkyl sulphanyl group is a group in which oxygen atoms of alkoxyl are replaced by sulfur atoms. The alkyl sulphanyl group is preferably C1-C20 alkyl sulphanyl group. The alkyl group in the alkyl sulphanyl group may or may not have a substituent.

The aryl ether group is preferably a C6-C40 aryl ether group, and further preferably a functional group of an aromatic hydrocarbon group bonded via an ether bond, such as phenoxyl group. The aryl ether group may or may not have a substituent.

The aryl thioether group is a group in which oxygen atoms of the ether bond of the aryl ether group are replaced with sulfur atoms. The aryl thioether group is C6-C60 aryl thioether group. The aromatic hydrocarbon group in the aryl thioether group may or may not have a substituent.

The aryl group is preferably C6-C60 aryl group, and further preferably one or more of aromatic hydrocarbon groups such as phenyl group, naphthyl group, biphenyl group, phenanthryl group, phenyl terphenyl group, or pyrenyl group. The aryl group may or may not have a substituent.

The heteroaryl group is preferably C4-C60 aromatic heterocyclic group, and further preferably one or more of furyl group, thienyl group, pyrrole group, benzofuranyl group, benzothienyl group, dibenzofuranyl group, dibenzothienyl group, pyridyl group, quinolinyl group, or the like. The aromatic heterocyclic group may or may not have a substituent.

The carbonyl group, the carboxyl group, the oxycarbonyl group, the carbamoyl group or the alkylamino group may or may not have a substituent. The number of carbon atoms of the alkylamino substituent is not particularly limited, but is usually in the range of 2 to 60.

The silanyl group is represented as a functional group having a bond to a silicon atom, such as a trimethylsilyl group, triethylsilyl group, dimethyl tert-butylsilyl group, or triphenylsilyl group. The silanyl group may or may not have a substituent. The number of carbon atoms of the silanyl group is not particularly limited, but is usually in the range of 1 to 40.

The above substituents are selected from one or more of the group consisting of deuterium, halogen, C1-C15 alkyl group, C3-C15 cycloalkyl group, C3-C15 heterocyclic group, C2-C15 alkenyl group, C4-C15 cycloalkenyl group, C2-C15 alkynyl group, C1-C55 alkoxyl group, C1-C55 alkyl sulphanyl group, C6-C55 aryl ether group, C6-C55 aryl thioether group, C6-C55 aryl group, C4-C55 aromatic heterocyclic group, carbonyl group, carboxyl group, oxycarbonyl group, carbamoyl group, C1-C55 alkylamino group or C3-C15 silanyl group with 1-5 silicon atoms.

The aromatic amine compound is not particularly limited, and examples may be specifically listed below.

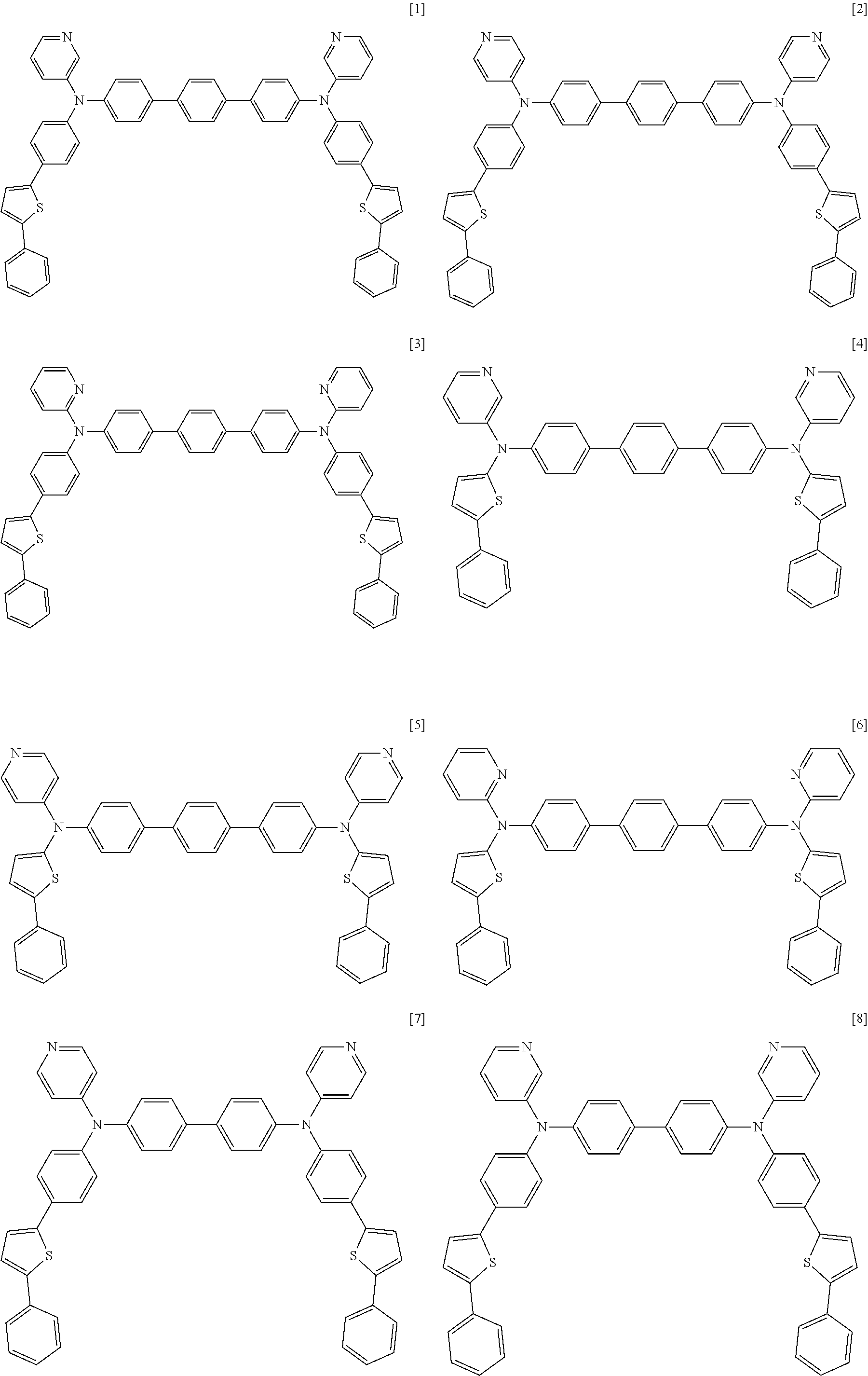

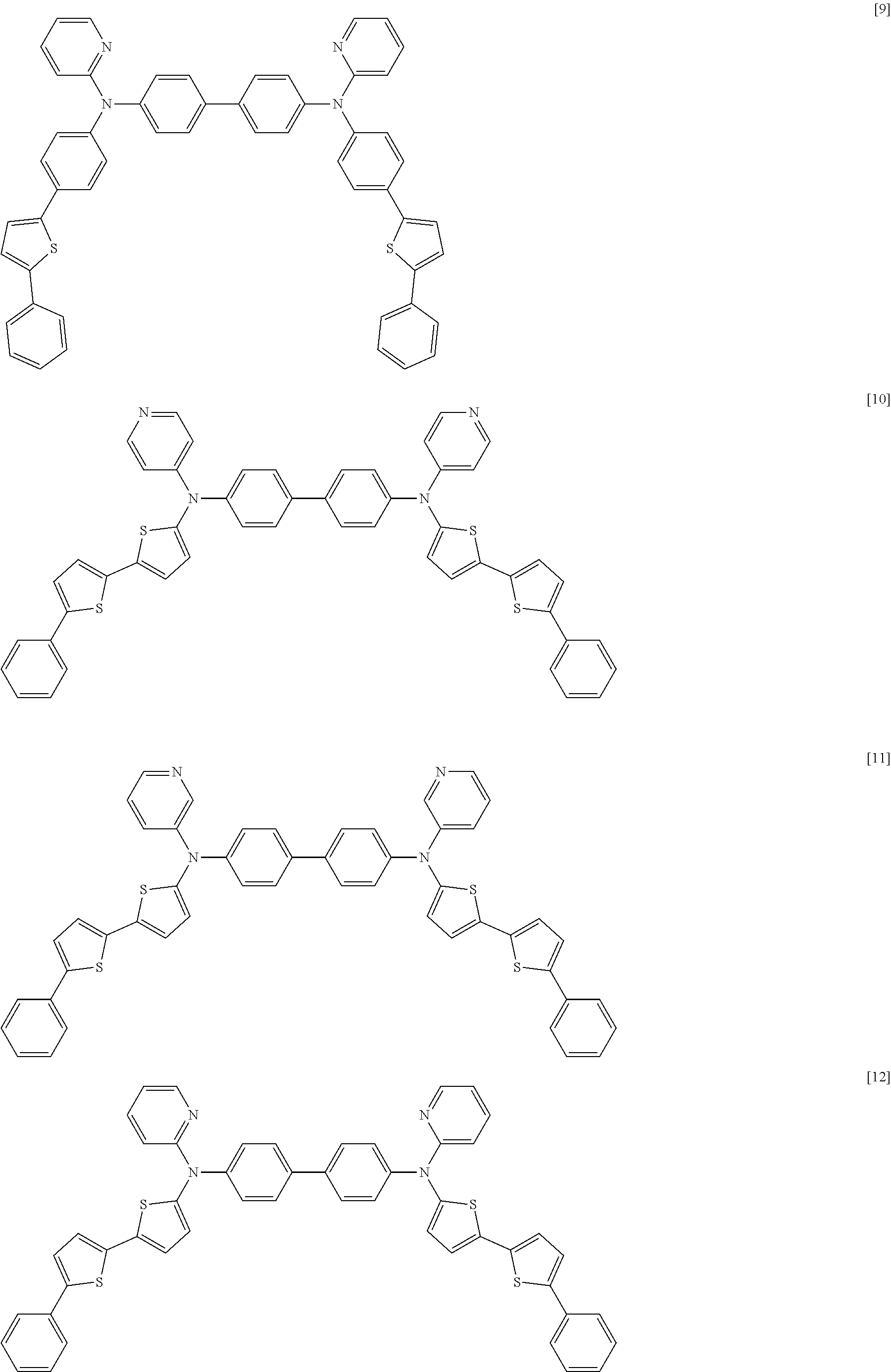 [SMILES: c1ccc(-c2ccc(-c3ccc(N(c4ccc(-c5ccc(N(c6ccc(-c7ccc(-c8ccccc8)s7)cc6)c6ccccn6)cc5)cc4)c4ccccn4)cc3)s2)cc1 ; c1ccc(-c2ccc(-c3ccc(N(c4ccncc4)c4ccc(-c5ccc(N(c6ccncc6)c6ccc(-c7ccc(-c8ccccc8)s7)s6)cc5)cc4)s3)s2)cc1 ; c1ccc(-c2ccc(-c3ccc(N(c4ccc(-c5ccc(N(c6cccnc6)c6ccc(-c7ccc(-c8ccccc8)s7)s6)cc5)cc4)c4cccnc4)s3)s2)cc1 ; c1ccc(-c2ccc(-c3ccc(N(c4ccc(-c5ccc(N(c6ccccn6)c6ccc(-c7ccc(-c8ccccc8)s7)s6)cc5)cc4)c4ccccn4)s3)s2)cc1]

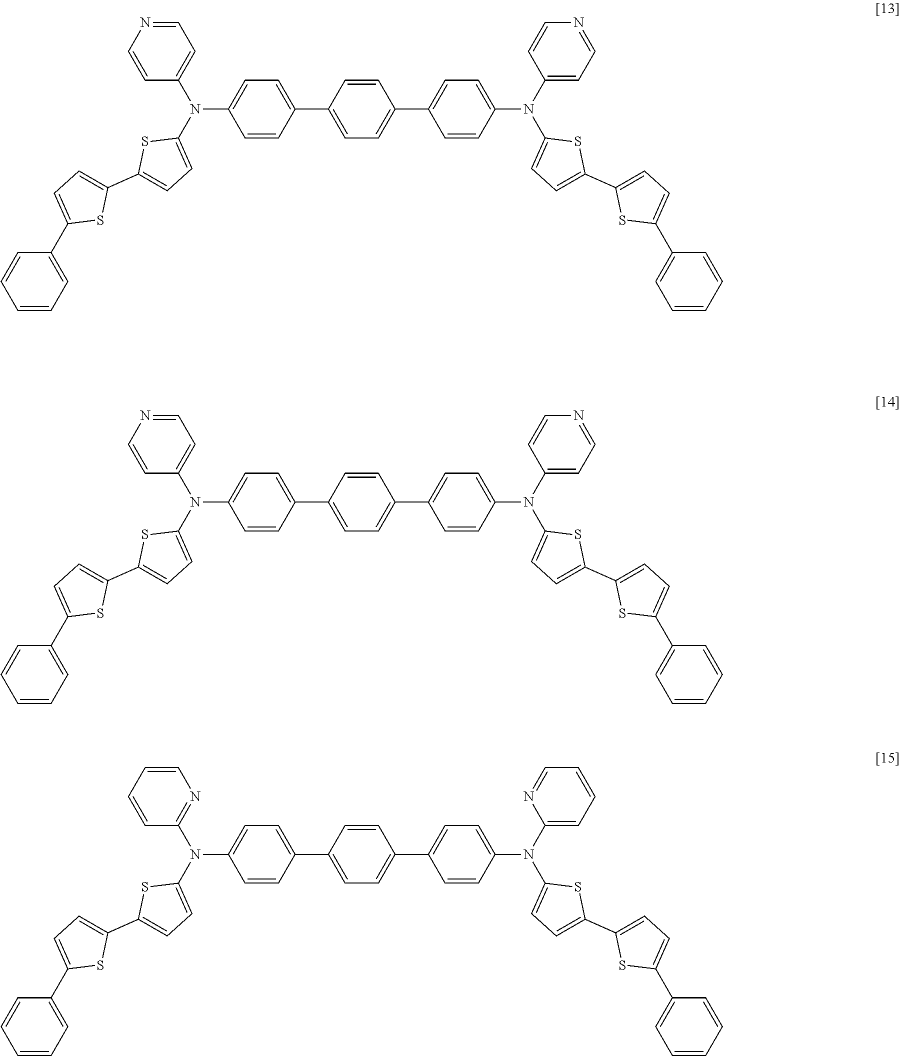 [SMILES: c1ccc(-c2ccc(-c3ccc(N(c4ccncc4)c4ccc(-c5ccc(-c6ccc(N(c7ccncc7)c7ccc(-c8ccc(-c9ccccc9)s8)s7)cc6)cc5)cc4)s3)s2)cc1 ; c1ccc(-c2ccc(-c3ccc(N(c4ccncc4)c4ccc(-c5ccc(-c6ccc(N(c7ccncc7)c7ccc(-c8ccc(-c9ccccc9)s8)s7)cc6)cc5)cc4)s3)s2)cc1 ; c1ccc(-c2ccc(-c3ccc(N(c4ccc(-c5ccc(-c6ccc(N(c7ccccn7)c7ccc(-c8ccc(-c9ccccc9)s8)s7)cc6)cc5)cc4)c4ccccn4)s3)s2)cc1]

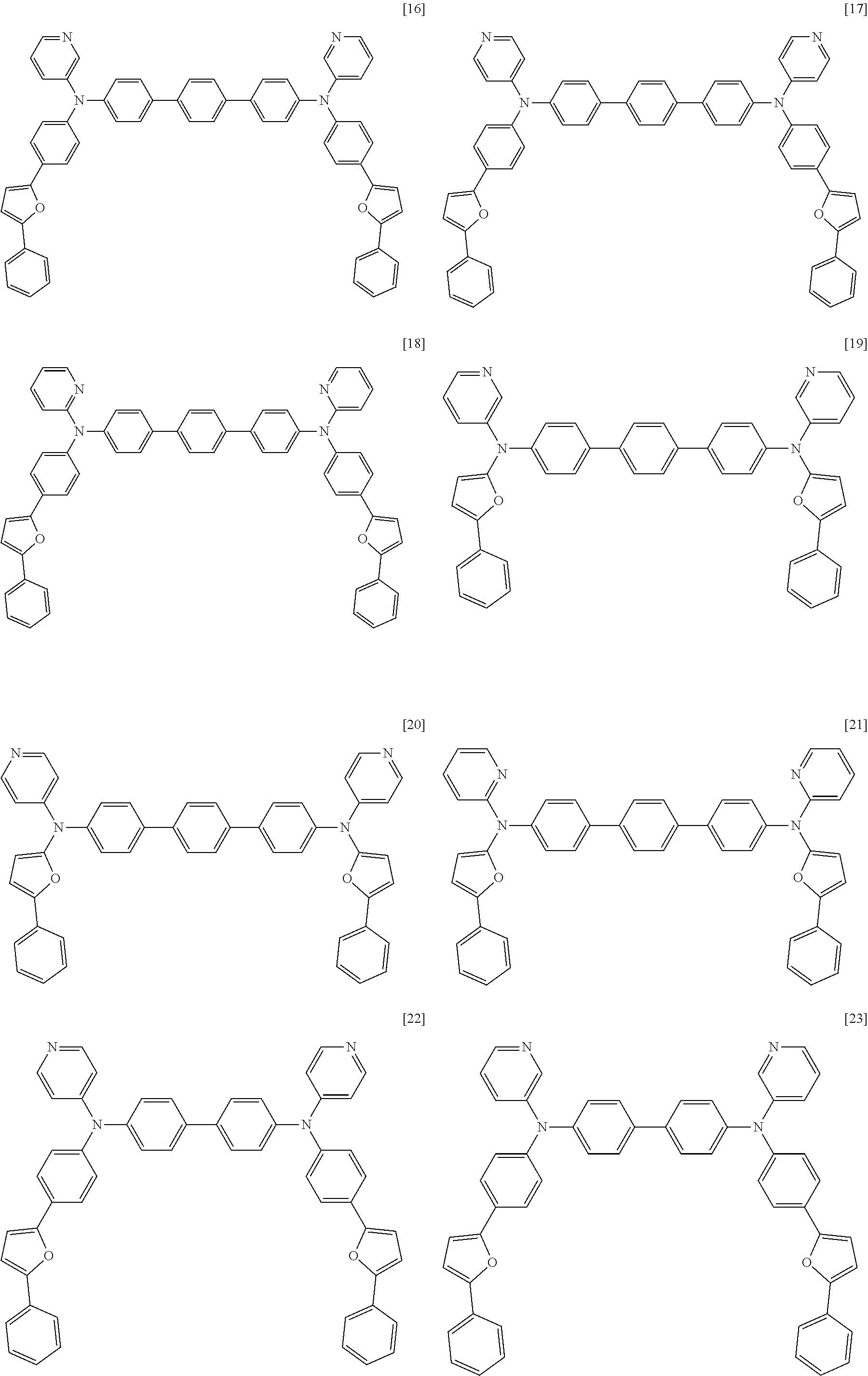

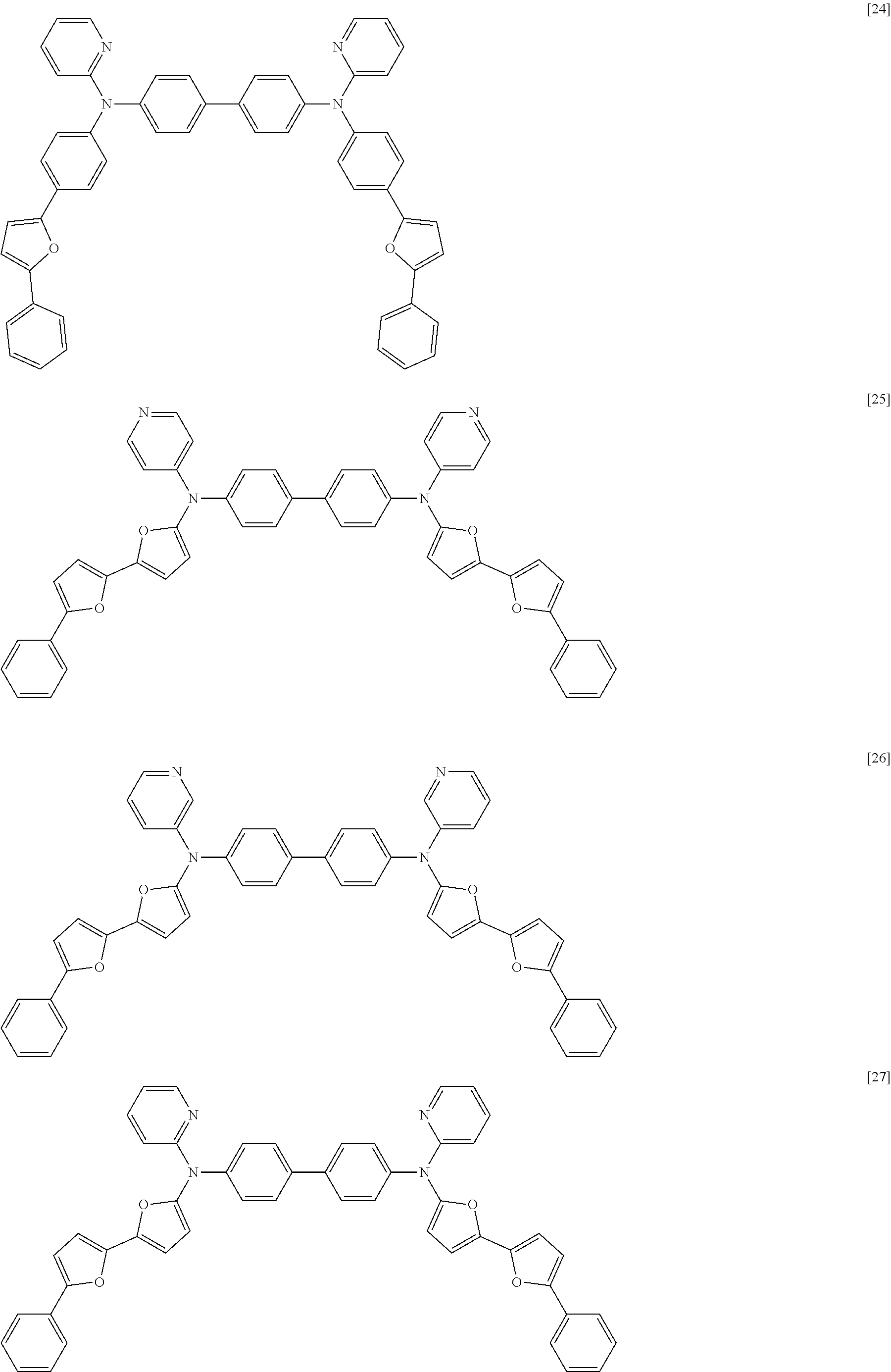 [SMILES: c1ccc(-c2ccc(-c3ccc(N(c4ccc(-c5ccc(N(c6ccc(-c7ccc(-c8ccccc8)o7)cc6)c6ccccn6)cc5)cc4)c4ccccn4)cc3)o2)cc1 ; c1ccc(-c2ccc(-c3ccc(N(c4ccncc4)c4ccc(-c5ccc(N(c6ccncc6)c6ccc(-c7ccc(-c8ccccc8)o7)o6)cc5)cc4)o3)o2)cc1 ; c1ccc(-c2ccc(-c3ccc(N(c4ccc(-c5ccc(N(c6cccnc6)c6ccc(-c7ccc(-c8ccccc8)o7)o6)cc5)cc4)c4cccnc4)o3)o2)cc1 ; c1ccc(-c2ccc(-c3ccc(N(c4ccc(-c5ccc(N(c6ccccn6)c6ccc(-c7ccc(-c8ccccc8)o7)o6)cc5)cc4)c4ccccn4)o3)o2)cc1]

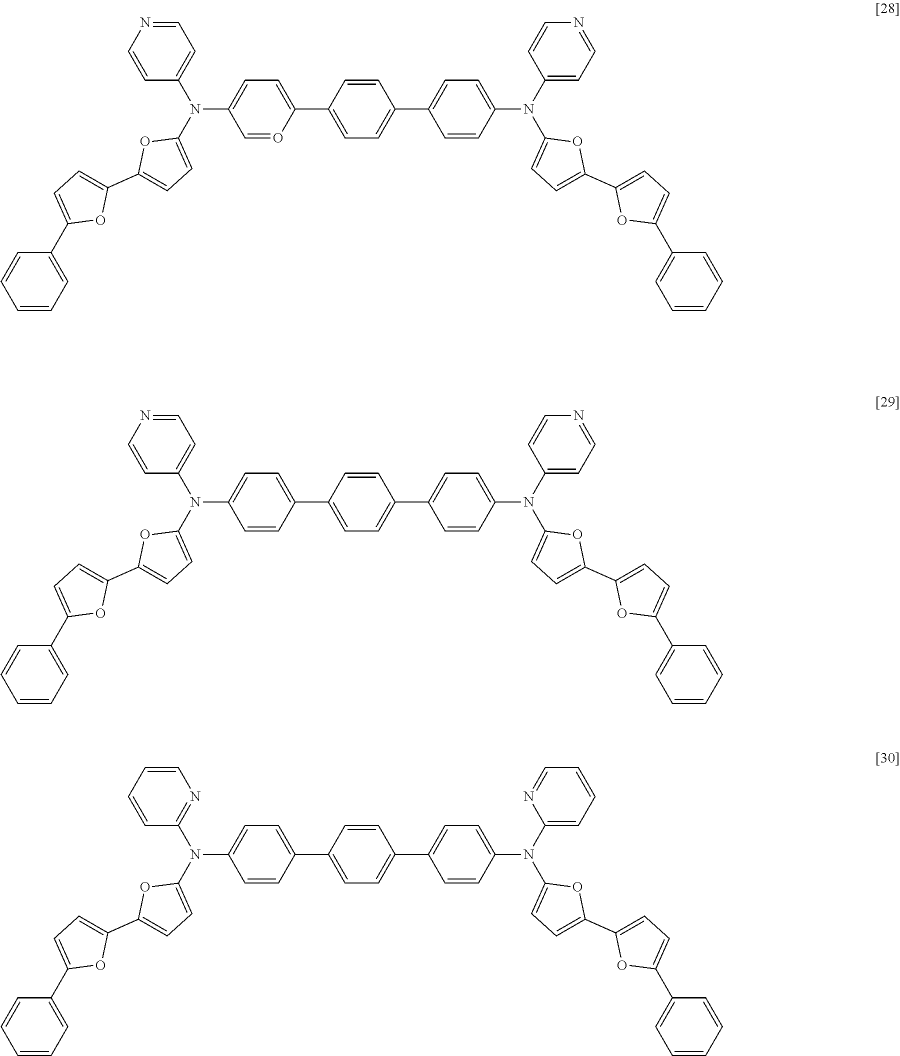

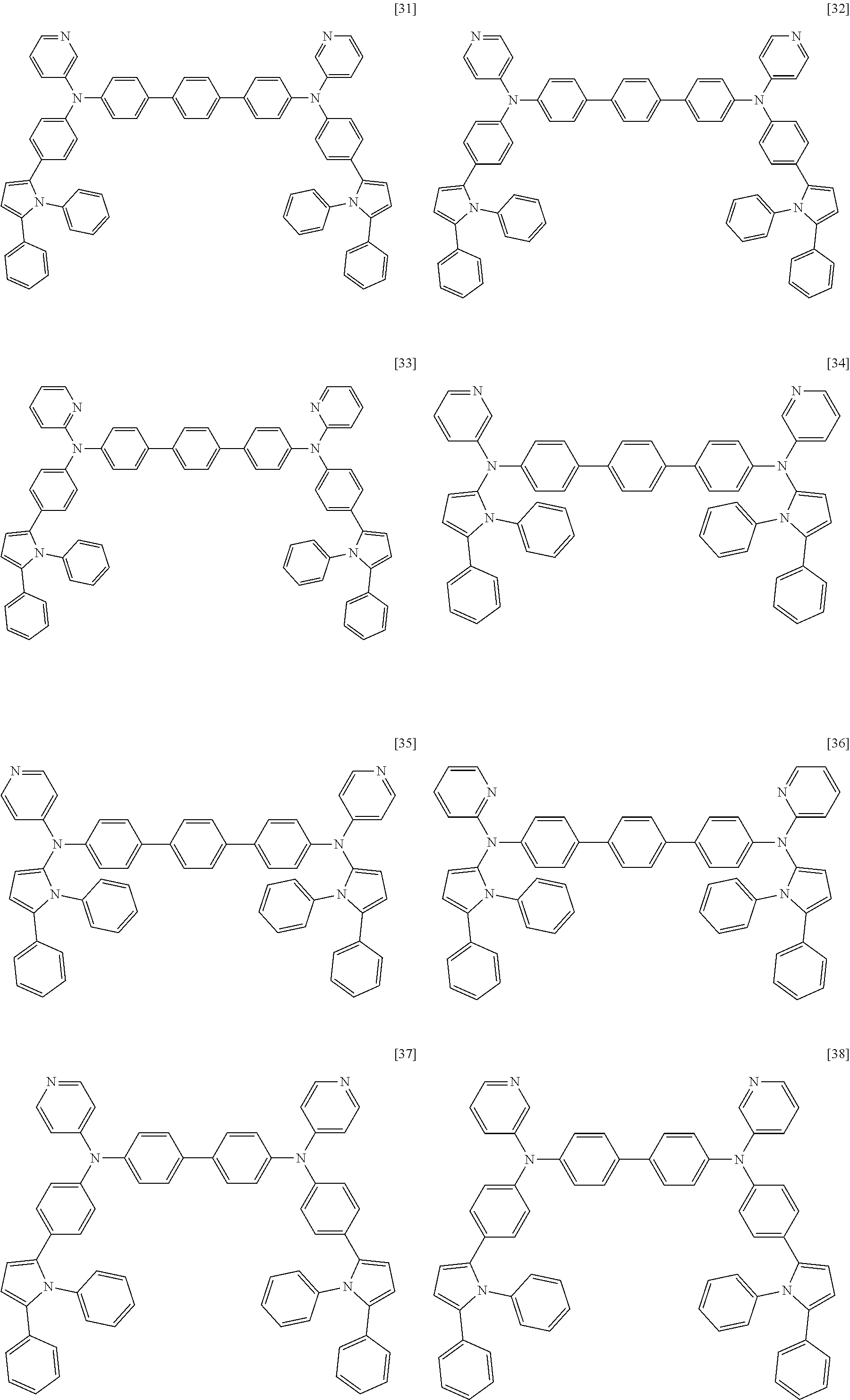

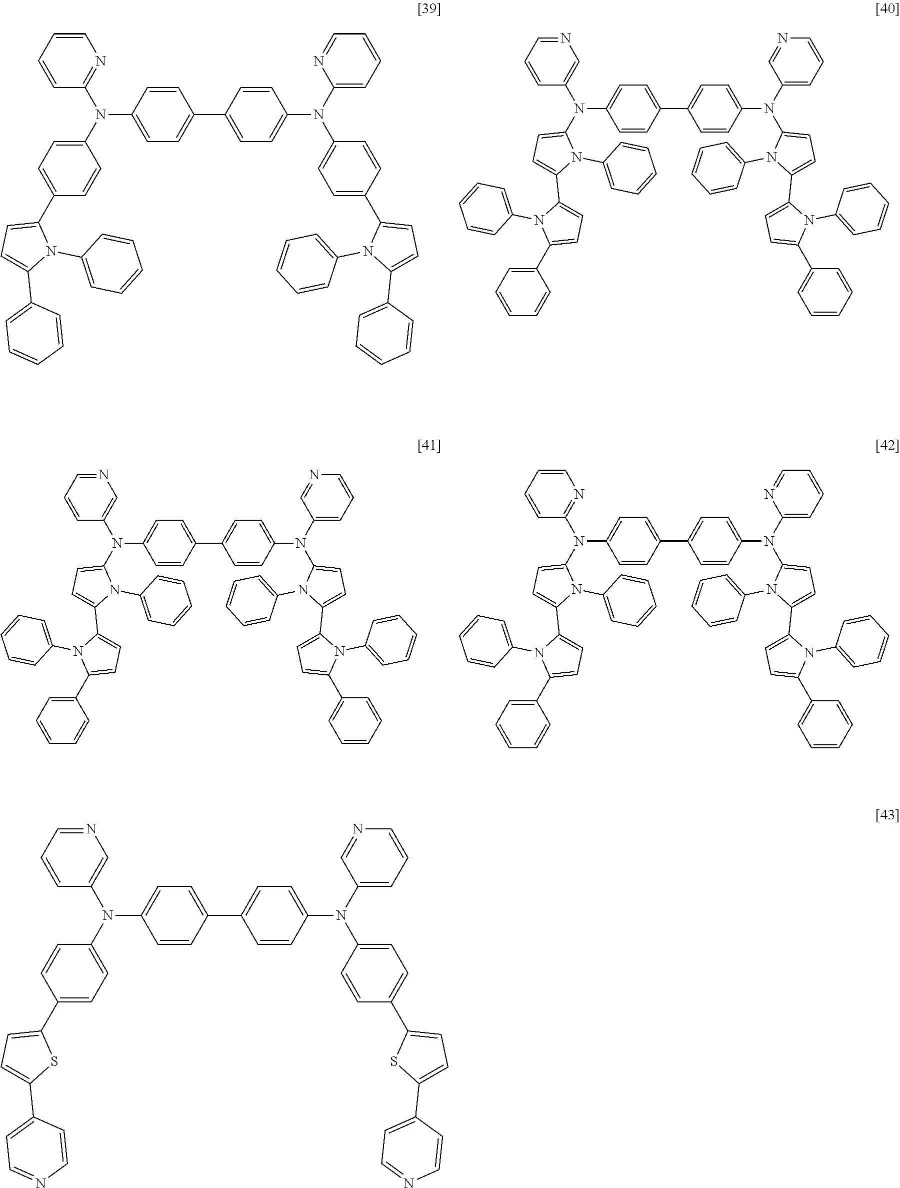

The synthesis of the aromatic amine compound represented by the formula (1) can be performed using known methods. For example, a Buchwald-Hartwig reaction using nickel or palladium and a Ullman reaction using copper are used, but not limited to these methods. In the above reactions, the Buchwald-Hartwig reaction is preferred in consideration of the characteristics of mild reaction conditions and excellent selectivity of various functional groups. In addition, when Ar² and Ar³ are different substituents, they are synthesized in stages according to a theoretical mixing ratio of amine to halide. The specific synthesis is shown in formula (2) below.

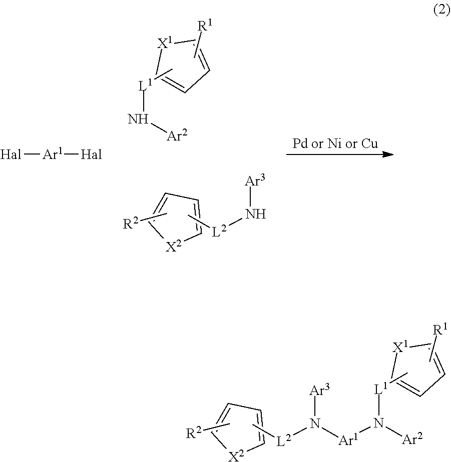

In the above formula (2), Hal represents halogen, such as a chlorine atom, a bromine atom or an iodine atom, or pseudo-halogen, such as a trifluoromethane sulfonate group.

The aromatic amine compound of the formula (1) in the present invention may be used alone or in combination with other materials in an organic light-emitting element.

Embodiments of the organic light-emitting element of the present invention will be specifically described hereinafter. The organic light-emitting element according to embodiments of the present invention is an organic light-emitting element containing the aromatic amine compound. The organic light-emitting element sequentially comprises a substrate, a first electrode, one or more organic layer films including a light-emitting layer, a second electrode through which light emitted from the light-emitting layer is transmitted, and a light extraction efficiency improving layer (i.e., the covering layer), wherein the light-emitting layer emits light by electric energy.

In the light-emitting element of the present invention, the adopted substrate is preferably a glass substrate, such as a soda glass substrate or an alkali-free glass substrate. As long as the thickness of the glass substrate is sufficient to maintain the mechanical strength, 0.5 mm or more is sufficient. As for the material of the glass, the less ions eluted from the glass, the better, and therefore, alkali-free glass is preferred. In addition, commercially available glass coated with protective coatings, such as SiO₂ or the like, can also be used. In addition, if the first electrode functions stably, the substrate may not necessarily be glass. For example, an anode may be formed on a plastic substrate.

The material used in the first electrode is preferably a metal, such as gold, silver, or aluminum having a high refractive index characteristic, or a metal alloy, such as an APC-based alloy. These metals or metal alloys may be laminated in multiple layers. In addition, transparent conductive metallic oxides, such as tin oxide, indium oxide, indium tin oxide (ITO), and indium zinc oxide (IZO), may be laminated on and/or under the metal, the metal alloy, or a laminate thereof.

The material used in the second electrode is preferably a material that can form a translucent or transparent film through which light may pass. For example, silver, magnesium, aluminum, and calcium, or alloys of these metals, and transparent conductive metallic oxides, such as tin oxide, indium oxide, indium tin oxide (ITO), or indium zinc oxide (IZO), may be used. These metals, alloys or metallic oxides may also be laminated in multiple layers.

The method for forming the electrodes may be resistance heating evaporation, electron beam evaporation, sputtering, ion plating, or a glue coating method, and is not particularly limited. In addition, according to a work function of the adopted material, one of the first electrode and the second electrode functions as an anode with respect to the organic film layer, and the other functions as a cathode.

The organic layer, besides being composed of a light-emitting layer only, may also be of a structure formed by laminating the following layers: 1) a hole transport layer/light-emitting layer; 2) a light-emitting layer/electron transport layer; 3) a hole transport layer/light-emitting layer/electron transport layer; 4) hole injection layer/hole transport layer/light-emitting layer/electron transport layer; or 5) a hole injection layer/hole transport layer/light-emitting layer/electron transport layer/electron injection layer, and the like. Furthermore, each of the above-mentioned layers may be any one of a single layer or a plurality of layers. When the structures 1) to 5) are adopted, the anode-side electrode is bonded to the hole injection layer or the hole transport layer, and the cathode-side electrode is bonded to the electron injection layer or the electron transport layer.

The hole transport layer can be formed by a method of laminating or mixing one or more than two kinds of hole transport materials, or a method using a mixture of a hole transport material and a polymer binder. The hole transport material is required to efficiently transfer holes from the anode between the electrodes to which an electric field is applied. Therefore, it is desirable that the hole injection efficiency is high and the injected holes can be efficiently transported. Therefore, the hole transport material is required to have an appropriate ionic potential, a large hole mobility, and further excellent stability, and is thus not easy to generate impurities that may become traps during manufacture and application. Substances that meet such conditions are not particularly limited. For example, such substances may be benzidines, such as 4,4′-bis(N-(3-methylphenyl)-N-phenylamino)biphenyl (TPD), 4,4′-bis(N-(1-naphthyl)-N-phenylamino)biphenyl (NPD), 4,4′-bis(N,N-bis(4-biphenyl)amino)biphenyl (TBDB), or bis(N,N-diphenyl-4-phenylamino)-N,N-diphenyl-4,4′-diamino-1,1′-biphenyl (TPD232); material groups called star-shaped triarylamine, such as 4,4′,4″-tris(3-methylphenyl(phenyl)amino)triphenylamine (m-MTDATA), 4,4′,4″-tris(1-naphthyl(phenyl)amino)triphenylamine (1-TNATA); materials having a carbazole structure, preferably carbazole-based polymers, specific examples including heterocyclic compound, such as, dicarbazole derivatives, for example bis(N-aryl carbazole) or bis(N-alkyl carbazole), tricarbazole derivatives, tetracarbazole derivatives, heterocyclic compounds such as triphenyl compounds, pyrazoline derivatives, stilbene compounds, hydrazine compounds, benzofuran derivatives, thiophene derivatives, oxadiazole derivatives, phthalocyanine derivatives and porphyrin derivatives, or fullerene derivatives. In the polymers, polycarbonates or styrene derivatives, containing the above-mentioned monomers in a side chain, polythiophene, polyaniline, polyfluorene, polyvinyl carbazole, polysilane, and the like, are also preferred. In addition, inorganic compounds, such as P-type Si and P-type SiC, may also be used.

The hole injection layer may be provided between the anode and the hole transport layer. By providing the hole injection layer, the organic light-emitting element may be enabled to achieve a low driving voltage and improve the durability. The hole injection layer is generally preferably made of a material having a lower ionic potential than the material of the hole transport layer. Specifically, the material of the hole injection layer may be, for example, the aforementioned benzidine derivative, such as TPD232, a star-shaped triarylamine material group, or a phthalocyanine derivative, or the like. In addition, it is also preferable that the hole injection layer is composed only of an acceptor compound or the acceptor compound is doped into other hole transport layers. Examples of the acceptor compound may include: metallic chlorides such as ferric trichloride (III), aluminum chloride, gallium chloride, indium chloride and antimony chloride, metallic oxides such as molybdenum oxide, vanadium oxide, tungsten oxide and ruthenium oxide, and charge transfer ligands such as tris(4-bromophenyl)ammoniumyl hexachloroantimonate (TBPAH). In addition, the acceptor compounds may be organic compounds, which contain nitro, cyano, halogen or trifluoromethyl in the molecules, quinone-based compounds, acid anhydride-based compounds, or fullerene, or the like.

In embodiments of the present invention, the light-emitting layer may be any of a single layer or multiple layers, and may be made of a light-emitting material (a host material and a doping material), respectively, and the light-emitting layer may be a mixture of a host material and a doping material, or may be only a host material, either of the above cases may be available. That is, in each light-emitting layer of the light-emitting element according to embodiments of the present invention, only the host material or only the doping material may emit light, or the host material and the dopant material may emit light together. From the viewpoint of efficiently utilizing electric energy and obtaining light with high color purity, it is preferable that the light-emitting layer is formed from a mixture of the host material and the doping material. In addition, the host material and the doping material may be a single material, or a combination of a plurality of materials, either of the above cases is possible. The doping material may be added to the entire host material, or added to a part of the host material, either of the above cases is possible. The doping material may be laminated, or be dispersed, either of the above cases is possible. The doping material may control the color of light emitted. When the doping material is excessive, a concentration extinction phenomenon occurs. Therefore, relative to the host material, the amount of the doping material is preferably 20% by weight or less, and more preferably 10% by weight or less. A doping method may be a method of co-evaporation with the host material, or a method of simultaneous evaporation after being mixed with the host material in advance.

As the light-emitting material, specifically, condensed ring derivatives such as anthracene and pyrene, which are conventionally known as light-emitting bodies, metal-chelating hydroxyquinoline compounds such as tris(8-hydroxyquinoline)aluminum, dibenzofuran derivatives, carbazole derivatives, indolocarbazole derivatives, and polymers, including polyphenylene vinylidene derivatives, poly(p-phenylene) derivatives, and polythiophene derivatives, etc., can be used, and are not particularly limited.

The host material contained in the light-emitting material is not particularly limited. Compounds having a condensed aromatic ring or derivatives thereof of such as anthracene, phenanthrene, pyrene, benzophenanthrene, tetracene, perylene, benzo[9,10]phenanthrene, fluoranthene, fluorene, indene, aromatic amine derivatives such as N,N′-dinaphthyl-N,N′-diphenyl-4,4′-diphenyl-1,1′-diamine, metal-chelating hydroxyquinoline compounds such as tris(8-hydroxyquinoline)aluminum, pyrrolopyrrole derivatives, dibenzofuran derivatives, carbazole derivatives, indolocarbazole derivatives, and triazine derivatives can be used. In the polymers, polyphenylene vinylidene derivatives, poly(p-phenylene) derivatives, polyfluorene derivatives, polyvinyl carbazole derivatives, polythiophene derivatives, or the like may be used, and will not be particularly limited.

In addition, the doping material is not particularly limited. Examples of the doping material may include: compounds having a condensed aromatic ring, such as naphthalene, anthracene, phenanthrene, pyrene, benzophenanthrene, perylene, benzo[9,10]phenanthrene, fluoranthene, fluorene and indene, or derivatives thereof (such as 2-(benzothiazole-2-yl)-9,10-diphenyl anthracene); and heteroaromatic ring-containing compounds, such as furan, pyrrole, thiophene, silole, 9-silafluorene, 9,9′-spirobisilafluorene, benzothiophene, benzofuran, indole, dibenzothiophene, dibenzofuran, imidazopyridine, phenanthroline, pyridine, pyrazine, naphthyridine, quinoxaline, pyrrolopyridine and thioxanthene, or derivatives thereof; borane derivatives, distyryl benzene derivatives, aminostyryl derivatives, pyrromethene derivatives, diketone pyrrolo[3,4-c]pyrrole derivatives, coumarin derivatives, azole derivatives, such as imidazole, triazole, thiadiazole, carbazole, oxazole, oxadiazole and triazole; and aromatic amine derivatives, and the like.

In addition, the light-emitting layer may be doped with a phosphorescent light-emitting material. The phosphorescent light-emitting material is a material that can phosphorescently emit light at room temperature. When the phosphorescent light-emitting material is used as a dopant, it is required to be capable of phosphorescently emitting light substantially at room temperature, but not be particularly limited. The phosphorescent light-emitting material is preferably an organometallic complex containing at least one metal selected from the group consisting of indium, ruthenium, rhodium, palladium, platinum, osmium, and rhenium. From the viewpoint of having high phosphorescent luminous efficiency at room temperature, an organic metal complex containing indium or platinum is more preferable. As a host material used in combination with the phosphorescent dopant, indole derivatives, carbazole derivatives, indolocarbazole derivatives, nitrogen-containing aromatic compound derivatives having pyridine-, pyrimidine-, triazine-structure; aromatic compound derivatives such as polyaryl benzene derivatives, spirofluorene derivatives, truxene and benzo[9,10]phenanthrene; compounds containing oxygen elements, such as dibenzofuran derivatives and dibenzothiophene, and organometallic complexes, such as hydroxyquinoline beryllium complexes, can be used well. Basically, it is not particularly limited as long as the triplet energy level of these materials is larger than that of the dopant used, and electrons and holes can be smoothly injected or transported from the respective transport layers. In addition, two or more triplet light-emitting dopants may be contained, and two or more host materials may also be contained. In addition, one or more triplet light-emitting dopants and one or more fluorescent light-emitting dopants may also be contained.

In the present invention, the electron transport layer is a layer in which electrons are injected from the cathode and then the electrons are transported. The electron transport layer should preferably have high electron injection efficiency and can efficiently transport the injected electrons. Therefore, the electron transport layer is preferably composed of a material which has large electron affinity and high electron mobility, excellent stability, and is less likely to generate impurities that can become traps during manufacture and use. However, in consideration of the transport equilibrium of holes and electrons, if the electron transport layer mainly plays a role that can efficiently prevent holes from the anode from flowing to the cathode side without being combined, even if the electron transport layer is composed of a material having less electron transport capability, the effect of improving the luminous efficiency is equivalent to that of a case in which a material having high electron transport capability is used. Therefore, in the electron transport layer of exemplary embodiments of the present invention, a hole barrier layer that can efficiently prevent hole migration is included as an equivalent.

The electron transport material used in the electron transport layer is not particularly limited. Examples of the electron transport material may include: condensed aromatic ring derivatives, such as naphthalene and anthracene; styryl-based aromatic ring derivatives represented by 4,4′-bis(diphenyl vinyl)biphenyl; quinone derivatives such as anthraquinone and biphenyl quinone; phosphine oxide derivatives; hydroxyquinoline complexes such as tris(8-hydroxyquinoline)aluminum; benzohydroxy quinoline complex, hydroxylazole complex, azomethine complex, tropolone metallic complex or flavonol metallic complex. It is preferable to use a compound having a heteroaromatic ring structure from the viewpoint of reducing the driving voltage and obtaining high-efficiency light emission. The heteroaromatic ring structure is composed of elements selected from carbon, hydrogen, nitrogen, oxygen, silicon, and phosphorus and containing electron-withdrawing nitrogen.

Heteroaromatic rings containing electron-withdrawing nitrogen have high electrophilicity. The electron transport material containing electron-withdrawing nitrogen easily accepts electrons from a cathode having high electrophilicity, and thus can reduce the driving voltage of the light-emitting element. In addition, since the supply of electrons to the light-emitting layer increases, the probability of recombination in the light-emitting layer increases, the luminous efficiency is improved. Examples of the heteroaromatic ring containing electron-withdrawing nitrogen include, for example: a pyridine ring, a pyrazine ring, a pyrimidine ring, a quinoline ring, a quinoxaline ring, a naphthyridine ring, a pyrimidopyrimidine ring, a benzoquinoline ring, a phenanthroline ring, an imidazole ring, an oxazole ring, an oxadiazole ring, a triazole ring, a triazole ring, a thiadiazole ring, a benzoxazole ring, a benzothiazole ring, a benzoimidazole ring, a phenanthroimidazole ring, or the like.

In addition, examples of the compounds having these heteroaromatic ring structures include, for example, low polypyridine derivatives such as benzimidazole derivatives, benzooxazole derivatives benzothiazole derivatives, oxadiazole derivatives, thiadiazole derivatives, triazole derivatives, pyrazine derivatives, phenanthroline derivatives, quinoxaline derivatives, quinoline derivatives, benzoquinoline derivatives, and other oligopyridine derivatives, such as bipyridine, terpyridine. When the above derivatives have a condensed aromatic ring structure, the glass transition temperature thereof is increased, and the electron mobility is improved. Therefore, the effect of reducing the driving voltage of the light-emitting element is improved, so it is thus preferable. In addition, it is preferred that the condensed aromatic ring structure is an anthracene-based structure, a pyrene-based structure, or a phenanthroline-based structure from the viewpoint of improving durability of the light-emitting device, easy synthesis, and easy purchase of raw materials.

The above-mentioned electron transport material may be used alone, or two or more kinds of the above-mentioned electron transport materials may be used in combination, or one or more other electron transport materials may be mixed into the above-mentioned electron transport materials. In addition, a donor compound may be added. Here, the donor compound refers to a compound that improves an electron injection energy barrier so that electrons can be easily injected from the cathode or the electron injection layer into the electron transport layer, thereby improving the electrical conductivity of the electron transport layer. Preferred examples of the donor compound of the present invention include: alkali metals, inorganic salts containing alkali metals, a complex of an alkali metal and an organic substance, alkaline earth metals, inorganic salts containing alkaline earth metals, or a complex of an alkaline earth metal and an organic substance. Preferred examples of the alkali metals or alkaline earth metals include: alkali metals such as lithium, sodium or cesium, or alkaline earth metals such as magnesium or calcium, which have a low work function and have a significant effect of improving the electron transport ability.

In embodiments of the present invention, an electron injection layer may be provided between the cathode and the electron transport layer. Generally, the electron injection layer is inserted to help inject electrons moving from the cathode to the electron transport layer. During insertion, a compound containing electron-withdrawing nitrogen and having a heteroaromatic ring structure or a layer containing the above-mentioned donor compound may be used. In addition, in the electron injection layer, an insulator or a semiconductor inorganic substance may be used. These materials are preferable because they can effectively prevent short-circuiting of the light-emitting element and improve the electron injection properties. As these insulators, at least one metallic compound selected from the group consisting of alkali metal chalcogenides, alkaline earth metal chalcogenides, alkaline metal halides, and alkaline earth metal halides is preferably used. In addition, a complex of organic substances and metals can also be used well.

Examples of the method for forming the above-mentioned layers constituting the light-emitting element include resistance heating evaporation, electron beam evaporation, sputtering, molecular lamination, or coating methods, and are not particularly limited. However, in general, from the viewpoint of element characteristics, resistance heating evaporation or electron beam evaporation is preferred.

The thickness of the organic layer varies depending on the resistance value of the light-emitting substance and it is not limited, but thickness of 1 to 1000 nm is preferable. The film thicknesses of the light-emitting layer, the electron transport layer, and the hole transport layer are preferably 1 nm or more and 200 nm or less, and more preferably 5 nm or more and 100 nm or less, respectively.

The light extraction efficiency improving layer (i.e., the covering layer) according to embodiments of the present invention contains the above-mentioned compound having a thiophene structure, a furan structure, or a pyrrole structure. In order to maximize the high luminous efficiency and achieve color reproducibility, it is preferable to laminate the compound having the thiophene structure, the furan structure or the pyrrole structure in a thickness of 20 nm to 120 nm. More preferably, the laminated thickness is 40 nm to 80 nm. In addition, from the viewpoint of maximizing the luminous efficiency, it is more preferable that the thickness of the light extraction efficiency improving layer (i.e., the covering layer) is more preferably 50 nm to 70 nm.

The method for forming the light extraction efficiency improving layer (i.e., the covering layer) is not particularly limited, and examples thereof include resistance heating evaporation, electron beam evaporation, sputtering, molecular lamination method, coating method, inkjet method, dragging method, and laser transfer printing method. The evaporation method is the most popular forming method. Substances that crystallize easily during this process will affect the overall performance of the device.

The light-emitting element of the present invention has a function of converting electric energy into light. Here, as the electric energy, a direct current is mainly used, but a pulse current or an alternating current may also be possible. The current and voltage values are not particularly limited, but when considering the power consumption and service life of the element, it should be selected in such a way that the maximum brightness can be obtained with the minimum energy.

For example, the light-emitting element of the present invention can be well used as a flat panel display that displays information in a mode of matrixes and/or fields.

The matrix mode means that the pixels used for display are arranged in a two-dimensional manner, such as grids or mosaic, and a set of pixels is used for displaying texts or images. The shape and size of the pixels depend on the applications. For example, in the image and text display of computers, monitors, and televisions, quadrilateral pixels with a side length 300 μm or less are usually used. In addition, in the case of a large-sized display, such as a display panel, pixels having a side length of mm-scale are used. In the case of monochromatic display, it is only necessary to arrange pixels of the same color, but in the case of color display, red, green, and blue pixels are arranged for display. In this case, a triangle type and a stripe type are typically used. Moreover, a driving method for the matrixes may be any one of a line-by-line driving method and an active matrix. Although the line-by-line driving method has a simple structure, there may be cases where the active matrix is excellent when considering the operation characteristics. Therefore, it needs to be flexibly used according to applications.

The field mode in the present invention refers to a mode in which a pattern is formed, and an area determined by the configuration of the pattern emits light to display predetermined information. Examples may include: time and temperature display in digital clocks and thermometers, display of working states of audio equipment, electromagnetic cookers, etc., and panel display of automobiles. Moreover, the matrix display and the field display may coexist in the same panel.

The light-emitting element of the present invention is preferably used as an illumination light source, and can provide a light source that is thinner and lighter than the existing light sources and that can perform surface light-emitting

### DETAILED DESCRIPTION OF EMBODIMENTS OF THE INVENTION

The aromatic amine compound of the present invention is exemplified by the following embodiments, but the present invention is not limited to the aromatic amine compounds and synthetic methods exemplified by these embodiments.

Toluene, xylene, methanol, 3-aminopyridine, and the like are commercially purchased from Sinopharm; 4,4′-dibromobiphenyl, 2-(4-bromophenyl)-5-phenylthiophene, and the like are commercially purchased from Zhengzhou Haikuo Optoelectronics Co., Ltd. Various palladium catalysts are commercially purchased from Aldrich Company.

¹H-NMR spectrum is measured using a JEOL (400 MHz) nuclear magnetic resonance instrument; a HPLC spectrum is measured using a Shimadzu LC-20AD high-performance liquid chromatography.

The following substances are used in the preparation examples, examples and comparative examples:

Compound [1]: 4,4″-bis(N-(3-pyridyl)-(4-(2-(5-phenylthienyl))phenyl)amine)yl-straight-chain terphenyl

Compound [4]: 4,4″-bis(N-(3-pyridyl)-2-(5-phenylthienyl)amine)yl-straight-chain terphenyl

Compound [8]: 4,4′-bis(N-(3-pyridyl)-(4-(2-(5-phenylthienyl))phenyl)amine)yl-biphenyl

Compound [23]: 4,4′-bis(N-(3-pyridyl)-(4-(2-(5-benzofuran))phenyl)amine)yl-biphenyl

Compound [38]: 4,4′-bis(N-(3-pyridyl)-(4-(2-(5-phenyl N-phenylpyrrole))phenyl)amine)yl-biphenyl

Compound [43]: 4,4′-bis(N-(3-pyridyl)-(4-(2-(5-phenyl N-phenylpyrrole))N-phenylpyrrole)amine)yl-biphenyl

Com-2: N,N,N′,N′-tetra(4-biphenyl)diamino biphenylene

NPD: N,N′-diphenyl-N,N′-bis(1-naphthyl)-1,1′-biphenyl-4,4′-diamine (the structure is as follows)

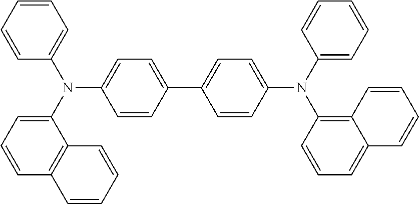 [SMILES: c1ccc(N(c2ccc(-c3ccc(N(c4ccccc4)c4cccc5ccccc45)cc3)cc2)c2cccc3ccccc23)cc1]

F4-TCNQ: 2,3,5,6-tetrafluoro-7,7′,8,8′-tetracyanodimethyl p-benzoquinone) (the structure is as follows)

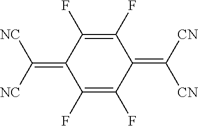 [SMILES: N#CC(C#N)=c1c(F)c(F)c(=C(C#N)C#N)c(F)c1F]

BH: (9-(2-naphthyl)-10-(4-(1-naphthyl)phenyl)anthracene (the structure is as follows)

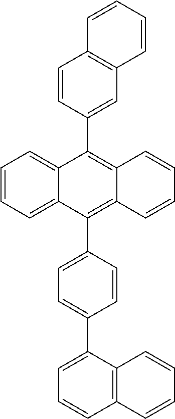 [SMILES: c1ccc2cc(-c3c4ccccc4c(-c4ccc(-c5cccc6ccccc56)cc4)c4ccccc34)ccc2c1]

BD: (E-7-(4-(diphenyl amino)styryl)-N,N-diphenyl-9,9′-dimethylfluorenyl-2-amine) (the structure is as follows)

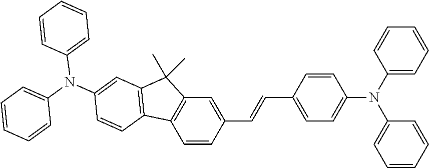 [SMILES: CC1(C)c2cc(/C=C/c3ccc(N(c4ccccc4)c4ccccc4)cc3)ccc2-c2ccc(N(c3ccccc3)c3ccccc3)cc21]

Alq₃: tris(8-hydroxyquinoline)aluminum (the structure is as follows)

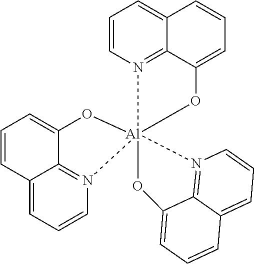

SPA: 2,5-bis(4-(N-(-3-biphenyl)-(N-3-pyridyl)aminophenyl)thiophene (the structure is as follows)

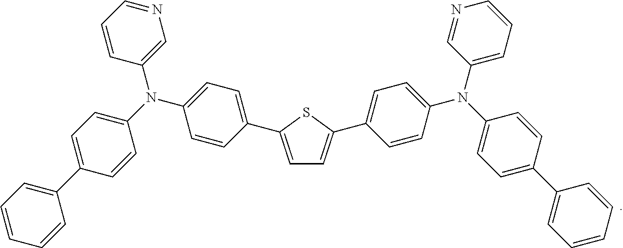 [SMILES: c1ccc(-c2ccc(N(c3ccc(-c4ccc(-c5ccc(N(c6ccc(-c7ccccc7)cc6)c6cccnc6)cc5)s4)cc3)c3cccnc3)cc2)cc1]

### Preparation Example 1

Synthesis of Compound [1]

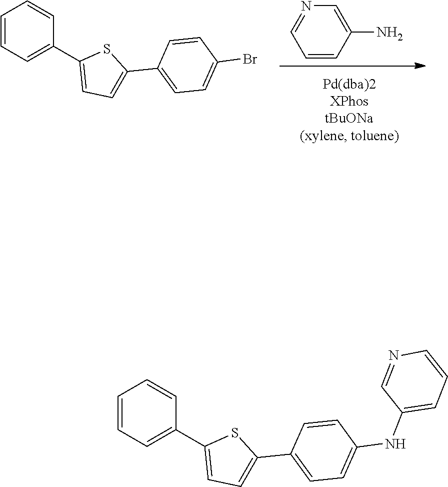 [SMILES: Brc1ccc(-c2ccc(-c3ccccc3)s2)cc1 ; Nc1cccnc1 ; c1ccc(-c2ccc(-c3ccc(Nc4cccnc4)cc3)s2)cc1]

In the presence of nitrogen, 2.07 g of 3-aminopyridine (22 mmol), 6.305 g of 2-(4-bromophenyl)-5-phenylthiophene (20 mmol), 4.61 g of sodium tert-butoxide (48 mmol), 0.23 g of bis(dibenzylidene)acetone)palladium (4.0 mmol), 0.38 g of 2-dicyclohexyl phosphonium-2′,4′,6′-triisopropyl biphenyl (8 mmol), 50 ml of toluene and 50 ml of xylene are added to a reactor, and stirred and refluxed for 6 hours. The reactant is filtered after being cooled to room temperature. A filter cake is rinsed with 100 ml of xylene, washed twice with 200 ml of water and filtered, washed while stirring twice with 200 ml of methanol and filtered, and vacuum-dried to obtain 5.6 g of [4-(5-phenyl-thiophen-2-phenyl]-3-pyridylamine.

¹HNMR (DMSO): δ8.52 (s, 1H), 8.22 (s, 1H), 8.12 (s, 1H), 7.88 (s, 2H), 7.48 to 7.41 (m, 3H), 7.32 to 7.22 (m, 6H), 6.54 to 6.51 (m, 2H)

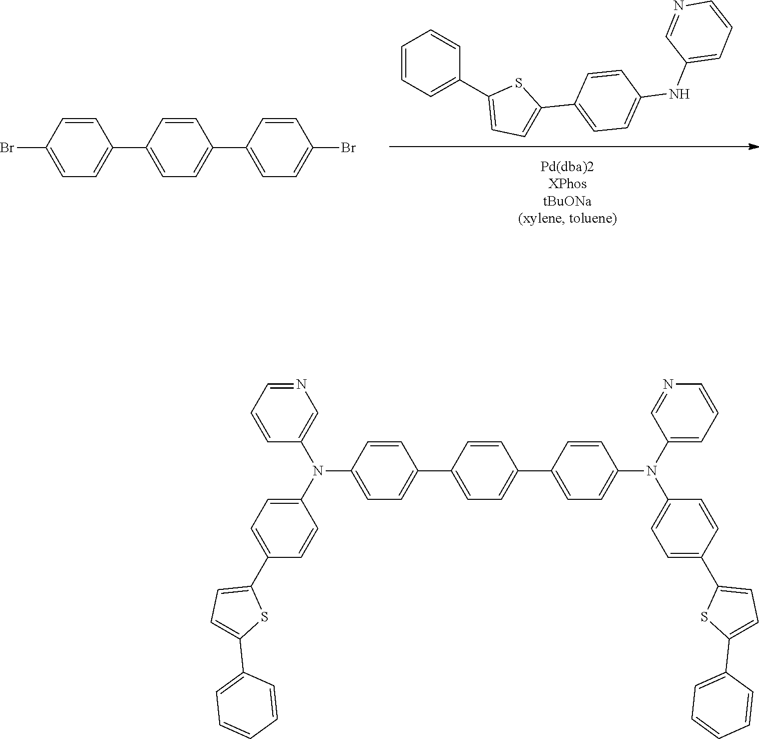 [SMILES: Brc1ccc(-c2ccc(-c3ccc(Br)cc3)cc2)cc1 ; c1ccc(-c2ccc(-c3ccc(Nc4cccnc4)cc3)s2)cc1 ; c1ccc(-c2ccc(-c3ccc(N(c4ccc(-c5ccc(-c6ccc(N(c7ccc(-c8ccc(-c9ccccc9)s8)cc7)c7cccnc7)cc6)cc5)cc4)c4cccnc4)cc3)s2)cc1]

In the presence of nitrogen, 5.25 g of [4-(5-phenyl-thiophen-2-phenyl]-3-pyridylamine (16 mmol), 3.10 g of 4,4′-dibromoterphenyl (8 mmol), 0.18 mg of bis(dibenzylidene)acetone)palladium (0.32 mmol), 0.30 mg of 2-dicyclohexyl phosphonium-2′,4′,6′-triisopropyl biphenyl (0.64 mmol), 1.85 g of sodium tert-butoxide (19.2 mmol), 60 ml of toluene and 60 ml of xylene are added to a reactor, heated, refluxed and stirred for 4 hours. The reactant is filtered after being cooled to room temperature. A filter cake is rinsed with 200 ml of xylene, washed with a mixture of 100 ml of water and 100 ml of methanol, then washed with 200 ml of water, filtered and dried to obtain 4.7 g of crude product. The crude product is sublimed at a pressure of 3×10⁻³ Pa and a temperature of 330° C. to obtain 2.4 g of the compound [1] (light yellow solid).

¹HNMR (CDCl₃): δ8.55 to 8.51 (s, 2H), 8.25 to 8.21 (m, 2H), 7.66 to 7.63 (s, 4H), 7.56 to 7.52 (m, 4H), 7.50 to 7.46 (m, 4H), 7.42 to 7.38 (m, 2H), 7.34 to 7.30 (m, 4H), 7.27 to 7.21 (m, 12H), 6.54 to 6.50 (m, 8H).

HPLC (purity=98.1%)

### Preparation Example 2

Synthesis of Compound [8]

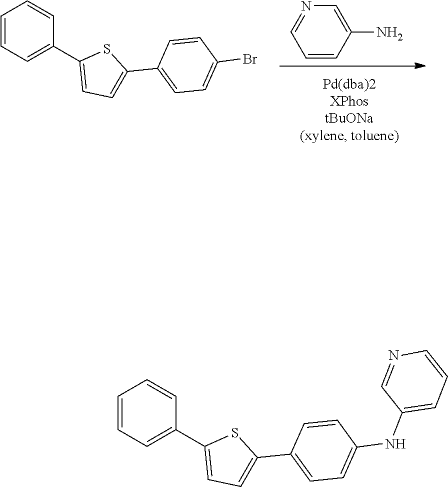 [SMILES: Brc1ccc(-c2ccc(-c3ccccc3)s2)cc1 ; Nc1cccnc1 ; c1ccc(-c2ccc(-c3ccc(Nc4cccnc4)cc3)s2)cc1]

In the presence of nitrogen, 2.07 g of 3-aminopyridine (22 mmol), 6.305 g of 2-(4-bromophenyl)-5-phenylthiophene (20 mmol), 4.61 g of sodium tert-butoxide (48 mmol), 0.23 g of bis(dibenzylidene)acetone)palladium (4.0 mmol), 0.38 g of 2-dicyclohexyl phosphonium-2′,4′,6′-triisopropyl biphenyl (8 mmol), 50 ml of toluene and 50 ml of xylene are added to a reactor, and stirred and refluxed for 6 hours. The reactant is filtered after being cooled to room temperature. A filter cake is rinsed with 100 ml of xylene, washed twice with 200 ml of water and filtered, washed while stirring twice with 200 ml of methanol and filtered, and vacuum-dried to obtain 5.6 g of [4-(5-phenyl-thiophen-2-phenyl]-3-pyridylamine.

¹HNMR (DMSO): δ8.52 (s, 1H), 8.22 (s, 1H), 8.12 (s, 1H), 7.88 (s, 2H), 7.48 to 7.41 (m, 3H), 7.32 to 7.22 (m, 6H), 6.54 to 6.51 (m, 2H)

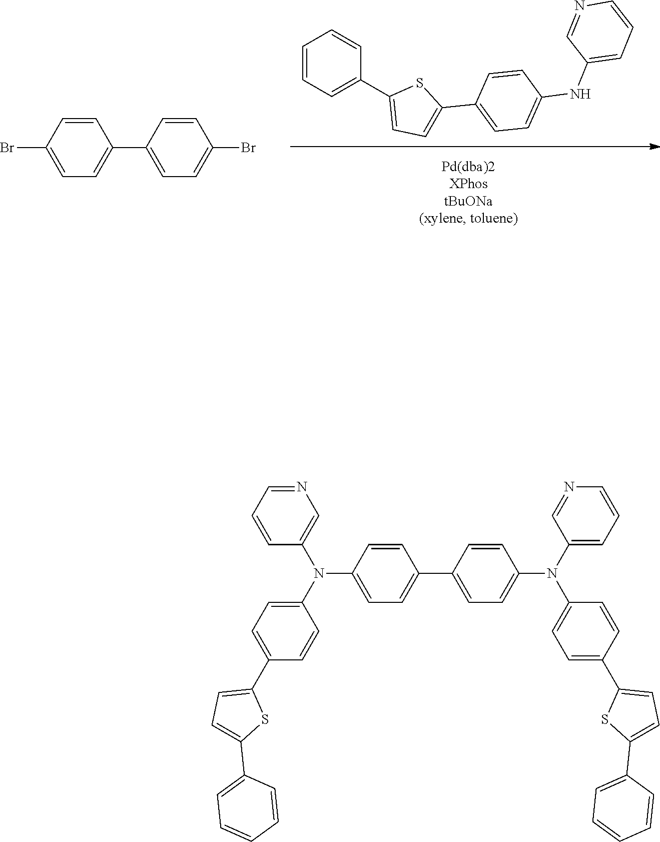 [SMILES: Brc1ccc(-c2ccc(Br)cc2)cc1 ; c1ccc(-c2ccc(-c3ccc(Nc4cccnc4)cc3)s2)cc1 ; c1ccc(-c2ccc(-c3ccc(N(c4ccc(-c5ccc(N(c6ccc(-c7ccc(-c8ccccc8)s7)cc6)c6cccnc6)cc5)cc4)c4cccnc4)cc3)s2)cc1]

In the presence of nitrogen, 5.25 g of [4-(5-phenyl-thiophen-2-phenyl]-3-pyridylamine (16 mmol), 2.5 g of 4,4′-dibromoterphenyl (8 mmol), 0.18 mg of bis(dibenzylidene)acetone)palladium (0.32 mmol), 0.30 mg of 2-dicyclohexyl phosphonium-2′,4′,6′-triisopropyl biphenyl (0.64 mmol), 1.85 g of sodium tert-butoxide (19.2 mmol), 60 ml of toluene and 60 ml of xylene are added to a reactor, and heated, refluxed and stirred for 4 hours. The reactant is filtered after being cooled to room temperature. A filter cake is rinsed with 200 ml of xylene, washed with a mixture of 100 ml of water and 100 ml of methanol, washed with 200 ml of water, filtered and dried to obtain 4.5 g of crude product. The crude product is sublimed at a pressure of 3×10⁻³ Pa and a temperature of 310° C. to obtain 2.2 g of the compound [12] (light yellow solid).

¹HNMR (CDCl₃): δ8.55 to 8.51 (s, 2H), 8.25 to 8.21 (m, 2H), 7.66 to 7.63 (s, 4H), 7.56 to 7.52 (m, 4H), 7.50 to 7.46 (m, 4H), 7.42 to 7.38 (m, 2H), 7.34 to 7.30 (m, 4H), 7.27 to 7.21 (m, 8H), 6.54 to 6.50 (m, 8H).

HPLC (purity=98.6%)

### Preparation Example 3

Synthesis of Compound [4]

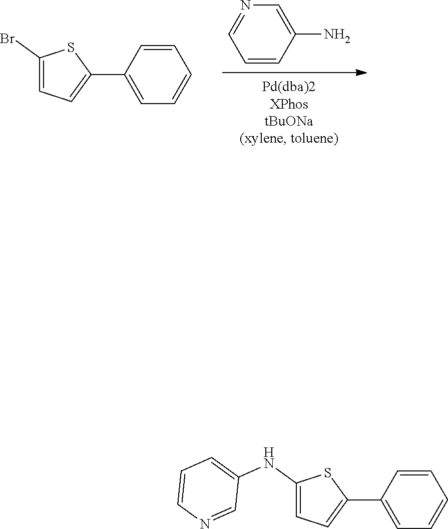 [SMILES: Brc1ccc(-c2ccccc2)s1 ; Nc1cccnc1 ; c1ccc(-c2ccc(Nc3cccnc3)s2)cc1]

In the presence of nitrogen, 2.07 g of 3-aminopyridine (22 mmol), 4.78 g of 2-bromo-5-phenylthiophene (20 mmol), 4.61 g of sodium tert-butoxide (48 mmol), 0.23 g of bis(dibenzylidene acetone)palladium (4.0 mmol), 0.38 g of 2-dicyclohexyl phosphonium-2′,4′,6′-triisopropyl biphenyl (8 mmol), 50 ml of toluene and 50 ml of xylene are added to a reactor, and stirred and refluxed for 6 hours. The reactant is filtered after being cooled to room temperature. A filter cake is rinsed with 100 ml of xylene, washed twice with 200 ml of water and filtered, washed while stirring twice with 200 ml of methanol and filtered, and vacuum-dried to obtain 4.3 g of [(5-phenyl-thiophen)]-3-pyridylamine.

¹HNMR (DMSO): δ8.52 (s, 1H), 8.22 (s, 1H), 8.12 (s, 1H), 7.48 to 7.41 (m, 3H), 7.32 to 7.22 (m, 4H), 6.54 to 6.51 (m, 2H)

HPLC (purity=98.1%)

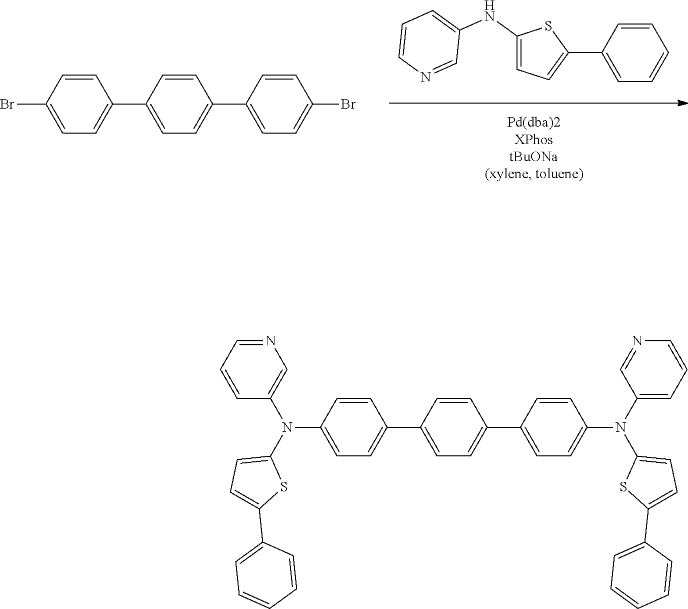 [SMILES: Brc1ccc(-c2ccc(-c3ccc(Br)cc3)cc2)cc1 ; c1ccc(-c2ccc(Nc3cccnc3)s2)cc1 ; c1ccc(-c2ccc(N(c3ccc(-c4ccc(-c5ccc(N(c6cccnc6)c6ccc(-c7ccccc7)s6)cc5)cc4)cc3)c3cccnc3)s2)cc1]

In the presence of nitrogen, 4.03 g of [(5-phenyl-thiophen]-3-pyridylamine (16 mmol), 3.10 g of 4,4′-dibromoterphenyl (8 mmol), 0.18 mg of bis(dibenzylidene acetone)palladium (0.32 mmol), 0.30 mg of 2-dicyclohexyl phosphonium-2′,4′,6′-triisopropyl biphenyl (0.64 mmol), 1.85 g of sodium tert-butoxide (19.2 mmol), 60 ml of toluene and 60 ml of xylene are added to a reactor, heated, stirred and refluxed for 4 hours. The reactant is filtered after being cooled to room temperature. A filter cake is rinsed with 200 ml of xylene, washed with a mixture of 100 ml of water and 100 ml of methanol, washed with 200 ml of water, and filtered and dried to obtain 3.6 g of crude product. The crude product is sublimed at a pressure of 3×10⁻³ Pa and a temperature of 300° C. to obtain 1.8 g of compound [4] (light yellow solid).

¹HNMR (CDCl₃): δ8.55 to 8.51 (s, 2H), 8.25 to 8.21 (m, 2H), 7.66 to 7.63 (s, 4H), 7.56 to 7.52 (m, 4H), 7.50 to 7.46 (m, 4H), 7.42 to 7.38 (m, 2H), 7.34 to 7.30 (m, 4H), 7.27 to 7.21 (m, 8H), 6.54 to 6.50 (m, 4H).

### Preparation Example 4

Synthesis of Compound [23]

Besides that 2-(4-bromophenyl)-5-phenylthiophene is replaced with 2-(4-bromophenyl)-5-phenylfuran, the rest is the same as that in Preparation Example 1. 2.3 g of compound [23] (white solid) is obtained.

¹HNMR (CDCl₃): δ8.54 to 8.51 (s, 2H), 8.25 to 8.21 (m, 2H), 7.67 to 7.63 (s, 4H), 7.56 to 7.52 (m, 4H), 7.50 to 7.46 (m, 4H), 7.43 to 7.38 (m, 2H), 7.34 to 7.30 (m, 4H), 7.27 to 7.21 (m, 12H), 6.55 to 6.51 (m, 8H).

HPLC (purity=98.5%)

### Preparation Example 5

Synthesis of Compound [38]

Besides that 2-(4-bromophenyl)-5-phenylthiophene is replaced with 2-(4-bromophenyl)-1,5-diphenyl-pyrrole, the rest is the same as that in Preparation Example 1. 2.6 g of compound [38] (white solid) is obtained.

¹HNMR (CDCl₃): δ8.55 to 8.52 (s, 2H), 8.25 to 8.21 (m, 2H), 7.67 to 7.63 (s, 4H), 7.56 to 7.52 (m, 4H), 7.50 to 7.46 (m, 4H), 7.43 to 7.38 (m, 2H), 7.35 to 7.29 (m, 12H), 7.25 to 7.21 (m, 12H), 6.53 to 6.50 (m, 8H).

HPLC (purity=98.7%)

### Preparation Example 6

Synthesis of Compound [43]

Besides that 2-(4-bromophenyl)-5-phenylthiophene is replaced with 4-[5-(4-bromobenzene)-2-thienyl]-pyridine, the rest is the same as that in Preparation Example 1. 2.0 g of compound [43] (white solid) is obtained.

¹HNMR (CDCl₃): δ8.55 to 8.51 (s, 2H), 8.25 to 8.21 (m, 2H), 7.66 to 7.63 (s, 4H), 7.56 to 7.52 (m, 4H), 7.50 to 7.46 (m, 4H), 7.42 to 7.38 (m, 2H), 7.34 to 7.30 (m, 4H), 7.27 to 7.21 (m, 10H), 6.54 to 6.50 (m, 8H).

HPLC (purity=98.3%)

### Example 1

Production Method of Thin Film Sample

An alkali-free glass substrate (Asahi Glass Co., Ltd., AN100) is subjected to UV ozone cleaning treatment for 20 minutes, further arranged in a vacuum evaporation apparatus and exhausted until the compound [12] is evaporated by a resistance heating evaporation method in the case that the vacuum degree in the apparatus is higher than 1×10⁻³ Pa, so as to prepare a thin film of about 50 nm. The evaporation rate is 0.1 nm/s.

The measurement of the refractive index and attenuation coefficient of the thin film sample prepared above is performed at Toray Research Center, Inc., and the adopted instrument is ellipsometric spectroscopy (J.A. Woollam Corporation M-2000).

TABLE 1

| | Refractive index (n) | | |
|---|---|---|---|
| Compound | λ = 430 nm | λ = 460 nm | λ = 500 nm |
| [8] | 2.52 | 2.33 | 2.13 |

Examples 2 to 6 and Comparative Examples 1, 2

### Example 2

Besides that the compound [8] is replaced with the compound [1], the rest is the same as that in Example 1.

The organic light-emitting element is evaluated. The evaluation results are shown in Table 2.

### Example 3

Besides that the compound [8] is replaced with the compound [4], the rest is the same as that in Example 1.

The organic light-emitting element is evaluated. The evaluation results are shown in Table 2.

### Example 4

Besides that the compound [8] is replaced with the compound [23], the rest is the same as that in Example 1.

The organic light-emitting element is evaluated. The evaluation results are shown in Table 2.

### Example 5

Besides that the compound [8] is replaced with the compound [38], the rest is the same as that in Example 1.

The organic light-emitting element is evaluated. The evaluation results are shown in Table 2.

### Example 6

Besides that the compound [8] is replaced with the compound [43], the rest is the same as that in Example 1.

The organic light-emitting element is evaluated. The evaluation results are shown in Table 2.

### Comparative Example 1

Besides that the compound [8] is replaced with NPD, the rest is the same as that in Example 1.

### Comparative Example 2

Besides that the compound [8] is replaced with SPA, the rest is the same as that in Example 1.

The organic light-emitting element is evaluated. The evaluation results are shown in Table 2.

TABLE 2

| | | Refractive index (n) | | |
|---|---|---|---|---|
| | Compound | λ = 430 nm | λ = 460 nm | λ = 500 nm |
| Example 2 | [1] | 2.50 | 2.31 | 2.12 |
| Example 3 | [4] | 2.48 | 2.30 | 2.12 |
| Example 4 | [23] | 2.48 | 2.32 | 2.12 |
| Example 5 | [38] | 2.50 | 2.30 | 2.09 |
| Example 6 | [43] | 2.51 | 2.32 | 2.13 |
| Comparative | NPD | 1.99 | 1.92 | 1.87 |
| example 1 | | | | |
| Comparative | SPA | 2.45 | 2.29 | 2.10 |
| example 2 | | | | |

As shown in Table 2, the refractive indexes of Examples 2 to 6 are significantly higher than those of Comparative Example 1. Further, the performances of the light-emitting element are tested.

Evaluation Method of Light-emitting Element

### Example 7

The alkali-free glass is ultrasonically washed in isopropyl alcohol for 15 minutes, and then subjected to UV ozone washing treatment in the atmosphere for 30 minutes. A vacuum evaporation method is used for evaporating 100 nm of aluminum as an anode and then sequentially laminate a hole injection layer (NPD and F4-TCNQ (weight ratio 97:3), 50 nm), a hole transport layer (NPD, 80 nm), a blue light-emitting layer (BH and BD (weight ratio 97:3, 20 nm), an electron transport layer (Alq₃, 30 nm), and an electron injection layer (LiF, 1 nm) by evaporation on the anode, and Mg and Ag (weight ratio 10:1, 15 nm) are then co-evaporated to obtain a translucent cathode.

Subsequently, the compound [8] (60 nm) is evaporated as a covering layer.

Finally, in a glove box with a dry nitrogen atmosphere, a sealing board made of alkali-free glass is sealed with an epoxy resin adhesive to produce a light-emitting element.

The above-mentioned light-emitting element is tested for brightness and color purity at room temperature and in the atmosphere by applying a direct current of 10 mA/cm² by means of a spectroradiometer (CS1000, Konica Minolta Co., Ltd.) for light emission from the sealing board. As measured according to the above-mentioned measured values, the photometric efficiency is 7.3 cd/A, and the color purity is CIE (x, y)=(0.139, 0.051). When the compound [8] is used as the covering layer, a high-performance light-emitting element with high light-emitting efficiency and high color purity is obtained.

The organic light-emitting element is evaluated. The evaluation results are shown in Table 3.

### Example 8

Besides that the covering layer material is the compound [1], the rest is the same as that in Example 7.

The organic light-emitting element is evaluated. The evaluation results are shown in Table 3.

### Example 9

Besides that the covering layer material is the compound [4], the rest is the same as that in Example 7.

The organic light-emitting element is evaluated. The evaluation results are shown in Table 3.

### Example 10

Besides that the covering layer material is the compound [23], the rest is the same as that in Example 7.

The organic light-emitting element is evaluated. The evaluation results are shown in Table 3.

### Example 11

Besides that the covering layer material is the compound [38], the rest is the same as that in Example 7.

The organic light-emitting element is evaluated. The evaluation results are shown in Table 3.

### Example 12

Besides that the covering layer material is the compound [43], the rest is the same as that in Example 7.

The organic light-emitting element is evaluated. The evaluation results are shown in Table 3.

### Comparative Example 3

Besides that the covering layer material is NPD, the rest is the same as that in Example 7.

The organic light-emitting element is evaluated. The evaluation results are shown in Table 3.

### Comparative Example 4

Besides that the covering layer material is SPA, the rest is the same as that in Example 7.

The organic light-emitting element is evaluated. The evaluation results are shown in Table 3.

TABLE 3

| | | Luminous | Color purity |
|---|---|---|---|
| | Compound | efficiency (cd/A) | CIE (x, y) |
| Example 7 | [8] | 7.3 | 0.139, 0.051 |
| Example 8 | [1] | 6.9 | 0.139, 0.051 |
| Example 9 | [4] | 6.6 | 0.138, 0.049 |
| Example 10 | [23] | 7.1 | 0.139, 0.050 |
| Example 11 | [38] | 7.0 | 0.139, 0.051 |
| Example 12 | [43] | 7.2 | 0.139, 0.051 |
| Comparative | NPD | 4.5 | 0.139, 0.048 |
| example 3 | | | |
| Comparative | SPA | 6.5 | 0.137, 0.051 |
| example 4 | | | |

As shown in Table 3, the light-emitting elements of Examples 7 to 12 satisfy both high light-emitting efficiency and high color purity. On the other hand, the light-emitting elements of Comparative Examples 3 to 4 have the same color purity as that in the examples, but have lower luminous efficiency than that in the examples. The light-emitting element in each example has higher luminous efficiency than that in Comparative Examples 3 and 4.

From the above results, it is derived that the aromatic amine compound according to embodiments of the present invention is suitable for organic light-emitting element materials to obtain the light-emitting element that satisfies high luminous efficiency and high color purity simultaneously, and is thus a more excellent covering layer material.

1. An aromatic amine compound comprising a structure represented by formula (1): 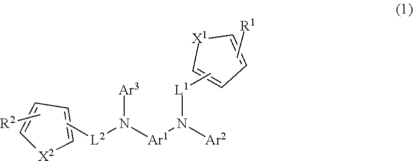
wherein, X¹ and X² are selected from a sulfur atom, an oxygen atom or N—R, wherein R is independently selected from one or more of the group consisting of hydrogen, deuterium, optionally substituted alkyl group, optionally substituted cycloalkyl group, optionally substituted heterocyclic group, optionally substituted alkenyl group, optionally substituted cycloalkenyl group, optionally substituted alkynyl group, optionally substituted alkoxyl group, optionally substituted alkyl sulphanyl group, optionally substituted aryl ether group, optionally substituted aryl thioether group, optionally substituted aryl group, optionally substituted heteroaryl group, optionally substituted carbonyl group, optionally substituted carboxyl group, optionally substituted oxycarbonyl group, optionally substituted carbamoyl group, optionally substituted alkylamino group, or optionally substituted silanyl group;
L¹ and L² may be identical or different, and independently selected from one of arylene group, heteroarylene group or direct bonding;
Ar¹ is selected from arylene group;
Ar² and Ar³ may be identical or different heteroaryl groups;
wherein, R¹ and R² may be identical or different, and are independently selected from one or more of the group consisting of hydrogen, deuterium, halogen, optionally substituted alkyl group, optionally substituted cycloalkyl group, optionally substituted heterocyclic group, optionally substituted alkenyl group, optionally substituted cycloalkenyl group, optionally substituted alkynyl group, optionally substituted alkoxyl group, optionally substituted alkyl sulphanyl group, optionally substituted aryl ether group, optionally substituted aryl thioether group, optionally substituted aryl group, optionally substituted heteroaryl group, optionally substituted cyano group, optionally substituted carbonyl group, optionally substituted carboxyl group, optionally substituted oxycarbonyl group, optionally substituted carbamoyl group, optionally substituted alkylamino group, or optionally substituted silanyl group; or may also be bonded with adjacent substituents to form a ring.

2. The aromatic amine compound according to claim 1, wherein, in formula (1), R¹, R² are one or more of aryl group or heteroaryl group.

3. The aromatic amine compound according to claim 1, wherein the X¹ and X² are selected from sulfur atoms; L¹ and L² are selected from arylene group; R¹ and R² are aryl group.

4. The aromatic amine compound according to claim 1, wherein the Ar¹ is non-condensed-ring aryl group.

5. The aromatic amine compound according to claim 1, wherein the Ar² and Ar³ are heteroaryl group directly connected to nitrogen.

6. The aromatic amine compound according to claim 1, wherein in formula (1), alkyl group is a C1-C20 alkyl group, cycloalkyl group is C3-C20 cycloalkyl group, heterocyclic group is C2-C20 heterocyclic group; alkenyl group is C2-C20 alkenyl group; cycloalkenyl group is C3-C20 cycloalkenyl group; alkynyl group is C2-C20 alkynyl group; alkoxyl group is C1-C20 alkoxyl group; alkyl sulphanyl group is C1-C20 alkyl sulphanyl group; aryl ether group is C6-C40 aryl ether group; the aryl thioether group is C6-C60 aryl thioether group; aryl group is C6-C60 aryl group; and heteroaryl group is C4-C60 aromatic heterocyclic group.

7. An organic light-emitting element material, wherein the material contains the aromatic amine compound according to claim 1.

8. An organic light-emitting element, comprising a substrate, a first electrode, a light-emitting layer containing one or more organic layer film, a second electrode, and a covering layer, wherein the organic light-emitting element contains the organic light-emitting element material according to claim 7.

9. A covering layer material of organic light-emitting element, wherein the material contains the aromatic amine compound according to claim 1.

10. An organic light-emitting element, comprising: a substrate, a first electrode, one or more organic layer film including a light-emitting layer, a second electrode, and a covering layer, wherein the covering layer contains the covering layer material of organic light-emitting element according to claim 9.
